(19) United States
(12) Patent Application Publication
(10) Pub. No.: US 2024/0127305 A1
(43) Pub. Date: Apr. 18, 2024

(54) PRE-SERVICE CLIENT NAVIGATION

(71) Applicant: Experian Health, Inc., Franklin, TN (US)

(72) Inventors: Edmond Chase Pilkington, Arrington, TN (US); Spiro E. Kalapodis, Valencia, CA (US); Ali Saffari, Diamond Bar, CA (US); Kristen Simmons, Aliso Viejo, CA (US); EIizabeth Donato Serie, Maple Grove, MN (US)

(21) Appl. No.: 18/344,588

(22) Filed: Jun. 29, 2023

Related U.S. Application Data
(63) Continuation of application No. 16/529,069, filed on Aug. 1, 2019, now abandoned.
(60) Provisional application No. 62/748,667, filed on Oct. 22, 2018.

(51) Int. Cl. G06Q 30/0601; G06N 20/00; G16H 10/00; G16H 40/00

(52) U.S. Cl. CPC G06Q 30/0611; G06N 20/00; G16H 10/00; G16H 40/00

(57) ABSTRACT

Aspects include a pre-service optimization system that provides timely and accurate estimate information to clients. Responsive to an indication of an order for a service, the system may generate an estimate of service costs, including the client responsibility amount, generate an elasticity estimate corresponding to the estimate including an indication of how likely the estimate is to change, a range within which the estimate is likely to change, and information about trigger points that affect the possible variability of the estimate. The system may push a notification to the client that includes a link to access the estimate and elasticity estimate. The notification may be delivered to the client in real time or near-real time as the estimate is generated. The system may further provide a user interface via which the client can access the estimate and elasticity estimate, and to access scheduling, payment, and financial assistance functionalities.

### CROSS-REFERENCE TO RELATED APPLICATIONS

This application claims the benefit of U.S. patent application Ser. No. 16/529,069, filed Aug. 1, 2019, titled “PRE-SERVICE CLIENT NAVIGATION,” and U.S. Provisional Application No. 62/748,667, filed Oct. 22, 2018, titled “Pre-Service User Navigation System,” the contents of which are incorporated by reference herein in their entirety.

### BACKGROUND

As prices of healthcare services and the patient financial responsibility for those healthcare services increase, and due in part to a lack of integrated data assets and consumerism, increasing strain is placed on the healthcare system. For example, patients oftentimes do not know how much their liability will be or their payment options. When an estimate is provided to a patient, that estimate is typically provided to the patient via an administrative user, and may be provided at the time of service. Due to various factors, estimates of service costs may change. Currently, if a patient receives an estimate, the patient may be unaware that the estimate may change, and may not be provided information about a level of likelihood that the estimate may change or why the estimate may change. As can be appreciated, patients may delay or avoid medically necessary procedures due to this lack of knowledge or for fear of going into debt, or may be unable or willing to pay for services after receiving services, and healthcare providers may increasingly hold onto larger accounts receivable for longer periods of time, as patients and their insurance providers delay in making payments for procedures, or even default on payments for procedures. Further, payment options or financial assistance options are typically not provided to patients prior to services being rendered; or if they are, they may be provided via separate systems.

An absence of providing patients with accurate estimate information prior to services being rendered places a strain on the healthcare system, in which healthcare providers may allocate additional processing resources to billing systems in effort to receive compensation. Additionally, a patient may determine whether to schedule a service based on an estimate, wherein a lack of an estimate, an inaccurate estimate, or an untimely estimate can cause patients to schedule services that they may later need to cancel, which requires additional processing of scheduling systems. Healthcare providers (or their agents) currently use various computer systems to perform various processes to provide healthcare services, and to bill for those services. These processes may include scheduling patients, finding patients' healthcare coverage, verifying patient demographic data, determining a patient financial history, estimating a charge for a procedure, and collecting payment for healthcare services, which may be performed by separate systems. As will be appreciated, if the speed or accuracy at which individual systems perform these processes were to be improved, or the capabilities of these individual systems were to be expanded, the overall system itself and the quality of care available to patients can be improved.

### SUMMARY

The present disclosure provides systems and methods for providing pre-service workflow optimization for improving client access to up-to-date and accurate estimate information and for improving the functionality of service access workflow systems, such as client access workflow systems used by healthcare providers to process patients. According to an aspect, responsive to receiving an indication of an order for or scheduling of a service for a client, a pre-service optimization system is configured to obtain data for determining an estimate of the cost of services and an estimate corresponding to the probability of the estimate changing and/or a range within which it may likely change (i.e., elasticity estimate), and to push a notification to the client that includes a link to a client navigation system that provides a user interface via which the client can view the estimate, view the elasticity estimate, and/or to access other pre-service workflow functionalities, such as scheduling functionalities, payment functionalities, financial assistance functionalities, and the like. For example, the client navigation system provides a user interface that guides the client through a pre-service workflow, which includes steps to finalize financial readiness prior to the service. The elasticity estimate is generated based on machine-learned rules that identify trigger points associated with various factors that affect the difference between the estimate for the upcoming service scheduled or ordered for the client and the actual service(s) provided to the client and a final cost of the service(s) that is ultimately billed to the client. The elasticity estimate informs the client of the probability or likelihood that the estimate will change, the amount by which it is likely to change by, and/or the factors that may influence the change. As can be appreciated, this information prepares the client for a probable fluctuation in service costs for which the client is responsible. This can increase the likelihood of the client receiving the service(s) and fulfilling the client responsibility.

According to another aspect, responsive to receiving a message from a service provider system or from a payer system, wherein the message is associated with an update of information corresponding to the upcoming service(s) for the client, another estimate is generated. If certain criteria are satisfied for notifying the client, a notification is pushed to the client that informs the client of the updated estimate. As can be appreciated, the notification is delivered to the client as data related to an upcoming service for the client is received and as the client's estimated client responsibility amount is determined/updated. Accordingly, the client is informed of a revised financial responsibility in real time or near-real time, which provides the client with the maximum amount of time to react to the revised estimate and to finalize financial readiness prior to the service. Accordingly, clients are less likely to default on payment of their responsibility amount. The pre-service optimization system simplifies and increases the efficiency of billing processes by pushing accurate estimate information to the client pre-service and guiding the client though steps (e.g., viewing an estimate, pre-paying for services, setting up a financial plan for upcoming services, determining whether the client qualifies for financial assistance, scheduling services) to finalize financial readiness prior to the service.

According to an aspect, the pre-service optimization system is configured to provide a single portal to various client access workflow process systems. Data provided to the user and actions taken by the user are synchronized with the client access workflow system, which simplifies the service provider's pre-service processes. For example, by pushing information to the user and providing a portal for enabling the user to finalize pre-service workflow processes, extra manual steps that may involve an administrative user interfacing with the user for providing an estimate, setting up a payment plan, applying the user for charity care, etc., can be eliminated.

In some aspects, the pre-service optimization system is configured to interface with a financial assistance system that determines whether the user is eligible to receive financial assistance or charity. If the user is eligible, the financial assistance system may be configured to automatically enroll the user into a financial assistance workflow.

In some aspects, the pre-service optimization system is configured to interface with a payment system, wherein the user is enabled to pay for services via the user interface.

In some aspects, the pre-service optimization system is configured to interface with a scheduling system, wherein the user is enabled to schedule a service or cancel a service for which an estimate is provided.

A reduction in the amount of communications and processing resources needed to offer financial assistance to users, when appropriate, is provided, which can further improve the efficiency of user access workflow systems.

### BRIEF DESCRIPTION OF THE DRAWINGS

The accompanying drawings, which are incorporated in and constitute a part of this disclosure, illustrate various aspects and examples of the present invention. In the drawings:

FIG. 1 is a block diagram illustrating an example operating environment for implementing aspects of the present disclosure;

FIG. 2 is a block diagram illustrating components of a pre-service optimization system with which aspects of the present disclosure may be practiced;

FIG. 3A illustrates an example notification pushed to a client;

FIGS. 3B-3L illustrate example client navigation user interfaces as may be displayed to a client for providing pre-service information and functionalities;

FIGS. 4A-4B is a flow chart showing general stages involved in an example method for performing pre-service workflow optimization functionalities as part of providing pre-service client financial navigation;

FIG. 5 is a block diagram illustrating physical components of an example computing device with which aspects may be practiced.

### DETAILED DESCRIPTION

The following detailed description refers to the accompanying drawings. Wherever possible, the same reference numbers are used in the drawings and the following description to refer to the same or similar elements. While aspects of the present disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the present disclosure, but instead, the proper scope of the present disclosure is defined by the appended claims. Examples may take the form of a hardware implementation, or an entirely software implementation, or an implementation combining software and hardware aspects. The following detailed description is, therefore, not to be taken in a limiting sense.

The present disclosure provides a system, method, and computer readable storage device including computer readable instructions, which when executed by a processing unit, provide pre-service workflow optimization for improving client access to up-to-date and accurate estimate information and for improving the functionality of client access workflow systems, such as the patient access workflow systems used by healthcare providers to process patients. Further, service-related information and tools are provided to clients in a unified user interface that improves the client's ability to navigate through pre-service workflow tasks for an improved service experience. Although examples are given herein primarily using a patient access workflow system and involving healthcare providers and patients, it will be recognized that the present disclosure is applicable to other types of services that provide estimates to clients. As such, the terms “user,” “patient,” and “client” may be used interchangeable herein.

Improvements to the speed, accuracy, and capabilities of these service access workflow systems not only improve the systems themselves, but can improve clients' access to services, such as a patient's access to healthcare services. For example, due to the complex nature of paying for healthcare services, which may include multiple payers (e.g., private insurance providers, governmental insurance providers, pharmaceutical companies, charities (including write-off and discount programs), guarantors, and the patients themselves) who have negotiated for different costs for a given procedure, the estimation of costs are handled by a pre-service optimization system that leverages data from multiple systems for generating and providing estimates and elasticity estimates with increased speed and accuracy.

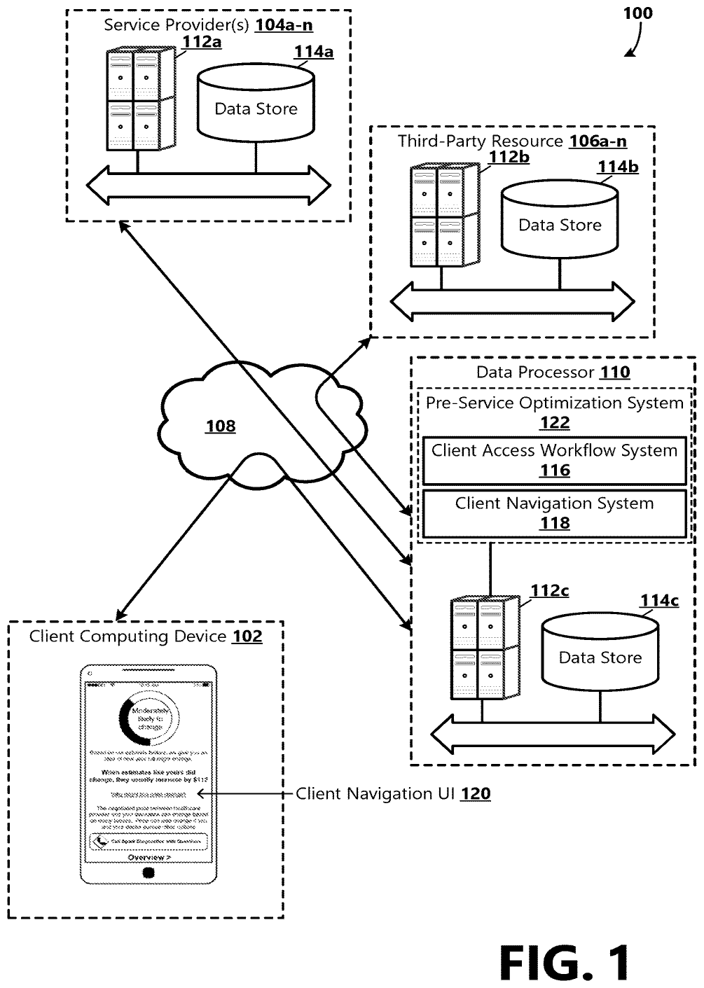

FIG. 1 is a block diagram illustrating an example operating environment 100 in which pre-service workflow optimization functionalities can be performed. In example aspects, systems within the example operating environment 100 exchange data and perform analytics to push delivery of service-related information, such as an estimate for an ordered or scheduled service, an estimate on the likelihood of that service estimate to change (i.e., estimate elasticity), and a real-time or near real-time notification if the service estimate changes, and service-related functionalities, such as a financial assistance screening tool, a payment interface, and a scheduling interface, to a client pre-service. Because these results may be used by parties when agreeing to or scheduling a course of treatment, and the fact that rendered healthcare services do not provide a security or other object that can be repossessed in the event of a default, the rendering of a cost estimate is a time-sensitive process, the speed and accuracy of which are of importance to both the patient and the healthcare provider. As used herein, “accuracy” and its related adjectives and adverbs do not refer to the correctness of how calculations are preformed (which are assumed to be performed correctly, unless stated otherwise), but refer to how close an estimate is to a final value. For example, if the final value for the costs of healthcare services is $100, an estimate of $99 would be more accurate than an estimate of $98.

With reference to FIG. 1, the example operating environment 100 includes a client computing device 102, one or more service provider systems 104a-n (generally, 104), a data processor system 110, and one or more third party resource 106a-n (generally, 106). Each of the systems 104, 106, 110 include one or more computing devices 112a-c (generally 112), include one or more data storage devices 114a-c (generally 114), and are in communication with a network 108 or a combination of networks for exchanging data and coordinating operations to push delivery of service-related information and functionalities to clients pre-service.

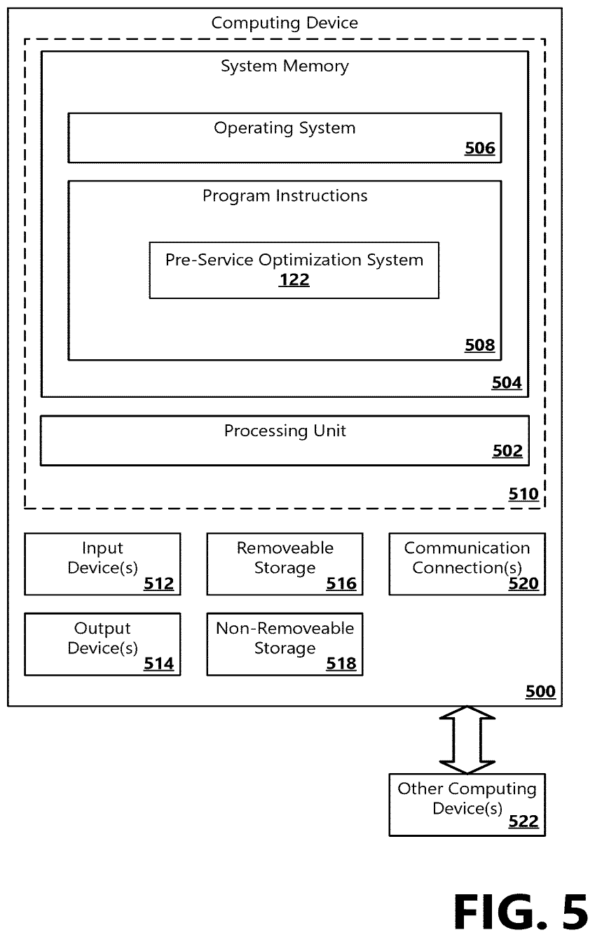

The one or more computing devices 112,102 are illustrative of a wide variety of computing devices, the hardware of which is discussed in greater detail in regard to FIG. 5. The client computing device 102 can be one of various types of computing devices. Non-limiting examples of client computing devices 102 include mobile devices, laptop computers, desktop computers, wearable computing devices, and other computing devices suitable to communicate with the data processor system 110. The client computing device 102 is configured to communicate with the data processor system 110 via the network 108. In some examples, the client computing device 102 includes a text messaging application, which is configured to receive text messages from senders, which can include a notification engine of the data processor system 110. In other examples, the client computing device 102 includes a client application, such as a mobile application that is installed on the device and is linked to a corresponding server-side application (e.g., notification engine) of the data processor system 110, wherein the client application is configured to receive push notification from the data processor system and/or to generate a local notification based on information received from the data processor system. The client can be the consumer receiving services from a service provider, a parent or guardian of the consumer, or any other person or entity responsible for the consumer's obligations regarding services rendered. The network 108 can be any type of public or private data network for communicating data between computer systems at different entities and at different geographic locations. The Internet is an example of one possible network 108.

The service provider system 104 is associated with a service provider that provides services to clients, such as a healthcare service provider that provides a healthcare service for which a client is ordered or scheduled to receive. According to an aspect, the service provider system 104 is configured to generate and transmit service-related messages to the data processor system 110, wherein the service-related messages include information related to a service or services ordered or scheduled for a client. For example, a physician may order or schedule a particular diagnostic procedure or test for a patient. When this procedure or test is entered by the physician (or an administrative user on behalf of the physician) into the service provider system 104, a service-related message may be automatically generated and transmitted by the service provider system 104 to the data processor system 110. The service-related message may include one or more one or more procedures anticipated to be performed, which may involve related procedures commonly performed in conjunction with the selected procedure. For example, a colonoscopy procedure may involve anesthesia, and in some cases, a biopsy, which would be included in the message data. Service-related messages may further include information about the client (e.g., client's name, account number, and other demographic data) and insurance information regarding the client's insurance coverage, such as, for example: whether the client has insurance coverage, and if so, who the insurance provider is, what type of plan the client has, etc. In example aspects, service-related message may be formatted according to particular formatting and protocol standards. For example, in a healthcare context, service-related messages may be formatted according to a set of standards for the electronic transfer of clinical and administrative data between software applications used by various healthcare providers, such as the Heath Level 7 (HL7) standard.

When a message is received by the data processor system 110, verifiers may be triggered to apply various rules and intelligence for verifying the received data against data in one or more data stores 114. According to aspects, the data processor system 110 includes a pre-service optimization system 122, which comprises a Client Access Workflow System (CAWS) 116 and a client navigation system (CNS) 118. Various requests may be sent automatically to the CAWS 116 to perform one or more client access workflow processes. An example of a CAWS 116 includes the eCare NEXT® platform, available from EXPERIAN HEALTH, INC. of Franklin, Tennessee. As one of ordinary skill in the art will appreciate, rules may be implemented via individual transistors that are discrete components of a circuit or via a processor that is configured by software to provide the corresponding logical operations necessary for comparison. In example aspects, the CNS 118 is configured to provide a user interface (client navigation UI 120) through which a client can receive an estimate for an ordered or scheduled service, estimate elasticity information, and updated service estimates when an estimate is updated. The CNS 118 may be further configured to provide, via the client navigation UI 120, access to a financial assistance screening tool for enabling the client to determine whether he/she may be eligible to receive financial assistance, a payment interface for enabling the client to pre-pay for or set up a payment plan for the ordered or scheduled service, and/or in some examples, a scheduling system interface for enabling the client to schedule an ordered service.

The third-party resource(s) 106 can include a variety of systems that the data processor system 110 may be configured to communicate with via the network 108. Examples of third party resources 106 can include payer (e.g., insurance company) systems, payment processing and/or payment gateway systems, scheduling systems, etc. In example aspects, a third party resource 106 may expose an API (Application Programming Interface) via which the data processor system 110 is enabled to receive or post information to third party resource or to integrate functionalities of third party resource into the UI (e.g., client navigation UI 120) provided by the data processor system 110. For example, the data processor system 110 may communicate with a payer system to obtain information related to benefits that a client is eligible to receive from the payer. As another example, the data processor system 110 may communicate with a payment processing or payment gateway system to enable clients to remit payment for a scheduled or ordered service. In some implementations, the data processor system 110 may direct the client to the payment processing or payment gateway system. As another example, the data processor system 110 may communicate with a scheduling system to enable clients to schedule or reschedule a service. Other types of third party resources 106 are possible and are within the scope of the present disclosure.

The data processor system 110 is in communication with the service provider system(s) 104, the third party resource(s) 106, and the client's computing device 102. The exchange of data and interaction between these systems of the example operating environment 100 are explained in more detail herein.

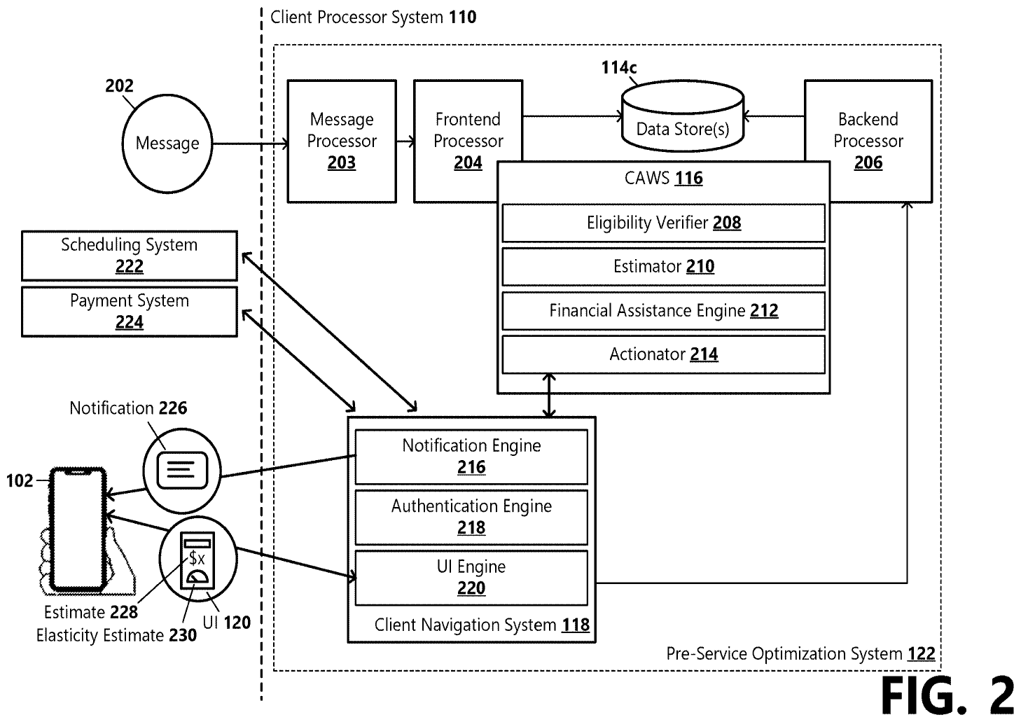

FIG. 2 is a block diagram illustrating an example embodiment of a pre-service optimization system 122 configured to provide pre-service workflow optimization. In the illustrated example, the pre-service optimization system 122 includes a message processor 203, frontend processor (FEP) 204, a backend processor (BEP) 206, a CAWS 116, a client navigation system 118, and a data store 114c. The data store 114c may be a single database or a plurality of databases. In an example aspect, the CAWS 116 may include an actionator 214 and various CAWS service engines, such as an eligibility verifier 208, an estimator 210, and a financial assistance system 212. As should be appreciated, other CAWS 116 services may be included. Instructions for the pre-service optimization system 122 can be executed by a single computing device 112. Alternatively, instructions for the pre-service optimization system 122 can be distributed across a plurality of computing devices that are in communication with each other and form a part of the pre-service optimization system 122.

The data store 114c is a hardware device that comprises computer readable storage media on which various information are stored. Additionally or alternatively, the data store 114c can include separate or secondary storage hardware in communication with the computing device executing the pre-service optimization system 122. In various examples, the data store 114c can be a cloud-based storage system that is separate and remote from the data processor system 110, but is in communication with the data processing system 110 through the network 108. As will be described in more detail below, the data store 114c may store: data conversion rules used by the message processor 203 for normalizing received messages 202; eligibility rules used by the eligibility verifier 208 for determining whether a client's eligibility as specified in a verification response is valid; conversion rules used by the eligibility verifier 208 for converting raw eligibility data into actionable information that can be used by the estimator 210 for generating an estimate 228; data and metadata such as training and historical data for training the (machine learning) estimator 210; rules used by the estimator for generating an estimate 228 for an ordered or scheduled service; service information including pricing information agreed to by service providers and payers for various services or diagnoses used by the estimator 210 for generating the estimate; rules used by the estimator for generating an elasticity estimate; the estimates and elasticity estimates generated by the estimator; business rules used by the actionator 214 for determining whether to notify a client about a determined estimate 228; client data about the client; and financial assistance screening rules for determining whether a client may qualify for financial assistance. The data store 114c may store additional data used for performing the above-mentioned and/or other pre-service workflow optimization functionalities.

According to aspects of the present disclosure, when a client seeks a service or services from a service provider, the pre-service optimization system 122 can be utilized to generate and provide an estimate 228 to the client (or to a guarantor of the client's account) pre-service so that an informed decision can be made regarding a course of treatment that includes the cost of that service. The estimate 228 may be determined by the estimator 210, which uses various information to determine the values in the estimate. According to an aspect, a triggering event for determining an estimate 228 can include a receipt of a message 202 from a service provider system 104, such as a hospital information system as would be found in a health management system. For example, the message 202 may include demographic information that identifies a client, a service that the client is requesting, and the service provider from which the client is requesting the service. As an example, in a healthcare context, the message can include, but is not limited to, the patient's name, the patient's date of birth, the patient's address(es), a patient account identifier, a healthcare provider identifier, and information about the service (e.g., what the service is, when the service is scheduled (if scheduled), where the service will be provided). As should be appreciated, the message 202 can include additional information, less information, or other combinations or types of information. Messages 202 may be formatted according to well-known formatting and protocol standards (e.g., Heath Level 7 or HL7) and/or electronic data interchange standards (e.g., X12), and may be sent to the pre-service optimization system 122 as single transactions or may be transmitted in a batch of messages. For example, a given batch file can include a plurality of ADT or X12 messages that may be passed from the service provider system 104 to the pre-service optimization system 122. In some implementations, the message 202 may be generated and transmitted to the pre-service optimization system 122 responsive to an order for the service being placed by the service provider. In other implementations, the message 202 may be generated and transmitted to the pre-service optimization system 122 responsive to an order for the service being scheduled by the service provider.

According to an aspect, the message 202 may be received by the message processor 203. For example, the message processor 203 is illustrative of a software application, module, or computing device operable or configured to receive messages 202 from the service provider system 104 and apply one or more data conversion rules stored in the data store 114c for converting the message data into a format such that message data can be used by other components of the pre-service optimization system 122. For example, the message 202 may be received in a first type of format, such as HL7, and the rules that are applied to the message may include encoding rules that convert the message data into another data format, such as XML (Extensible Markup Language) data, wherein an XML data file may use custom tags to define objects and the data within each object.

According to an aspect, the message processor 203 may be further configured to evaluate the normalized message data based on estimate criteria rules stored in the data store 114c, wherein the estimate criteria rules are configured to enable the message processor 203 to evaluate the normalized message 202 for making a determination as to whether the message 202 meets certain criteria for running an estimate 228. The estimate criteria rules 116 may be configurable and defined by each service provider. For example, a set of estimate criteria rules may be stored in the data store 114c for each service provider system 104 that is in communication with the pre-service optimization system 122. In some examples, the criteria are associated with the type of service ordered or scheduled for the client, the payer (e.g., commercial payer or self-pay versus a government assistance based program), or other factors. According to an aspect, when a message 202 is determined to satisfy the criteria corresponding to a set of estimate criteria rules, a determination may be made to run an estimate 228 for the service or services associated with the message 202. Responsive to determining to run an estimate for the message 202, the message processor 203 may be further configured to route the message 202 to the FEP 204.

In example aspects, the FEP 204 is illustrative of a software application, module, or computing device operable or configured to receive the message 202 from the message processor 203. According to an aspect, the FEP 204 may be configured to create an event, which operates as a trigger to one or more pre-service optimization system 122 components to perform certain operations for determining an estimate 228 and an elasticity estimate 230 for a service or services associated with the message 202.

According to an aspect, a rule may be applied to automatically match the normalized message data to client data stored in the data store 114c. For example, the message data may be matched to a client record based on the client's name or a unique identifier. In example aspects, client data may include data collected by and received from the service provider system 104, such as data collected as part of a registration process for the client and data produced as part of providing services to the client. For example, client data can include demographic data about the client, such as but not limited to: first name, middle name/initial, last name, address, telephone number(s) (e.g., including a mobile phone number that can be used to for delivery of text messages), email address, birthday, age, race, ethnicity, social security number, marital status, employer information, spouse information, etc. Client data can further include health-related information, such as but not limited to: patient type (e.g., outpatient, inpatient, emergency department, urgent care), healthcare coverage information (e.g., payer information), healthcare providers involved in the user's care, etc. In some implementations, client data can include additional patient- and provider-reported health data, such as information associated with diagnoses, medications, family medical history, lab and test results, biometric data, treatment history, etc. Other types of user data are possible and are within the scope of the present disclosure.

As part of matching the normalized message 202 data to client data stored in the data store 114c, the FEP 204 may identify a payer for the client. For example, the FEP 204 may be configured to match a client record to the client identified in the message 202 and to identify a payer (e.g., insurance company) included in the client record and other payer-related information (e.g., subscriber information, policy information). According to an aspect, a rule may be applied to automatically send a request to the eligibility verifier 208 to verify insurance coverages that the client may have based on the identified payer and payer-related information. The payer-related information and message data associated with the service or services that the client is seeking may be provided with or in the request.

The eligibility verifier 208 is illustrative of a software application, module, or computing device configured to perform medical eligibility verification, government assistance program eligibility verification, insurance eligibility verification, and health insurance eligibility verification. As part of performing eligibility verification, the eligibility verifier 208 may be configured to receive the request including the message data and payer information from the FEP 204, and to generate an eligibility verification request. In example aspects, the eligibility verifier 208 is configured to create a HIPAA (Health Insurance Portability and Accountability Act) compliant file (e.g., Healthcare Eligibility, Coverage and Benefit Inquiry (270)) for use within the context of an Electronic Data Interchange (EDI) environment for inquiring about the eligibility, coverages, or benefits associated with the client under an associated subscriber's policy. According to an aspect, the eligibility request (e.g., 270 inquiry) includes client data and message 202 data corresponding to the client in which eligibility detail is being requested, and can include service dates of the client, his/her identifying information, and service coding specific to the type of service ordered or scheduled to be received.

The eligibility verifier 208 may be further configured to transmit the eligibility request to a third-party system 106 corresponding to the payer identified in the client record, and to receive a Healthcare Eligibility, Coverage and Benefit Response (271 response) from the payer system that includes details of coverage, benefits, and eligibility. For example, the client's eligibility impacts the amount of payment for services, which accordingly impacts an estimate 228 for the services. The eligibility response from the payer may include but is not limited to: benefit status, explanation of benefits, coverages, effective dates, amounts for co-insurance, co-pays, deductibles, exclusions, and limitations. The eligibility response may be returned to the eligibility verifier 208, wherein the eligibility verifier is configured to receive the response and to apply eligibility rules for determining whether the client's eligibility is valid. In some implementations, the eligibility verifier 208 may be further configured to apply conversion rules to convert raw eligibility data provided by the payer system into actionable information that can be used by the estimator 210 for generating an estimate 228. According to an aspect, the eligibility verifier 208 may communicate the eligibility data to the BEP 206, wherein the BEP 206 may store the eligibility information in the data store 114c.

According to an aspect, a rule may be applied that instructs the FEP 204 to compile the normalized message data and the eligibility information and to pass the compiled data to the estimator 210. The estimator 210 is illustrative of a software application, module, or computing device operative or configured to generate an estimate 228 for the service or services included in the message 202, wherein the estimate includes a value associated with a total cost for providing the service(s) and out-of-pocket expenses that will be the responsibility of the client after payment is received from a third party payment source (i.e., payer). For example, a rule may be applied to automatically calculate the estimate 228 based on costs associated with the service(s) less insurance and any discounts. In an example aspect, the costs associated with the service(s), referred to herein as service information, may be based on specific prices agreed to by the service provider and the payer for particular services or diagnoses. The service information may be provided to the pre-service optimization system 122 as part of the client eligibility benefits data obtained from the client's payer, or may be stored in the data store 114c and accessed by the estimator 210 for generating the estimate 228.

According to another aspect, a rule may be applied to automatically calculate an elasticity estimate 230 associated with the estimate 228, wherein the elasticity estimate corresponds to a probability or likelihood that the estimate 228 will change and an amount by which the estimate 228 is likely to change. The estimator 210 is operable or configured to use one or more models to determine the estimate 228 and elasticity estimate 230, wherein the one or more models may be trained based on historical data. For example and as will be described in further detail below, the historical data may include data for clients who received services from the service provider. The historical data may include input data, calculated estimates data, remit data, and claim data for the clients. Accordingly, the historical data may reveal trends or relationships between various client input data, estimated amounts for services, and actual provided services and amounts billed for those services. When an estimate 228 and an elasticity estimate 230 are determined by the estimator 210, the estimator may be further configured to send the estimate 228 and the elasticity estimate 230 to the BEP 206, which stores the estimate and elasticity estimate in the data store 114c.

According to an aspect, the estimator 210 comprises a learner component illustrative of a software application, module, or computing device operative or configured to execute a machine-learning algorithm against learning data (including historical data) to generate (i.e., learn) rules that can be understood and used by the estimator 210 model(s) to analyze and make decisions corresponding to efficiently and accurately identifying trigger points associated with changes to estimates 228 and effects of those trigger points on the estimates. In example aspects, trigger points are associated with various factors that affect the difference between one or more estimates 228 of a service or services that a client is seeking and the actual service(s) provided to the client and a final cost of the service(s) that is ultimately billed to the client or a guarantor of the client.

One example trigger point includes a type of service provided to the client. As an example, the estimator 210 is configured to use a machine-learning algorithm to learn, based on historical data, that a first type of service (e.g., cataract procedure) can be estimated within a certain percentage of accuracy (e.g., 85% accuracy) and/or within a certain dollar range (e.g., within $115 of final cost), wherein a different type of service (e.g., a baby delivery) can be estimated within a lower percentage of accuracy or within a wider dollar range due to various conditions associated with the service that can affect changes to the services actually provided and the probability of those conditions occurring (e.g., conditions that may cause a patient to deliver a baby via an emergency Cesarean Section procedure versus a planned vaginal delivery). As can be appreciated, the costs associated with an emergency Cesarean Section procedure versus a planned vaginal delivery can vary by a wide dollar range due to differences in the length of stay at the service provider facility, surgical procedures that may be involved, additional medications that may be administered, etc. Other trigger points may include a place of service, a payer (e.g., insurance company), an insurance plan, equipment used, supplies used and received, a procedure used, etc. These trigger points are not limiting to the vast number of possible trigger points that can be learned via analyses of learning data comprised of training data and/or historical data collected from past analyses and decisions made by the estimator and end results associated with those decisions (e.g., determined estimates 228 for a service or services, the actual service(s) provided, the final costs of providing the actual service(s), and the client's responsibility for those final costs). According to an aspect, regression analysis may be performed on the data within the estimator 210 model to estimate relationships among the various types of historical data. For example, one or more formulas may be generated based on the regression analysis that represent the estimated relationship between client data, computed estimates 228, and actual final costs. The trained estimator 210 model may then be implemented to determine a predicted elasticity estimate 230 for an estimate 228 for a client by leveraging the relationships determined by the regression analysis.

The learning data (e.g., training data and/or historical data) may be stored in the data store 114c. Learning data can include positive examples that indicate when a desired result has been achieved. For example, a desired result may be when a final cost for providing a service is within a certain percentage or dollar amount of accuracy to an estimate 228 that was determined for that service. Another example desired result may be when an estimate 228 changes and the change is within a certain percentage or dollar amount of accuracy to an elasticity estimate 230 that was previously determined for that estimate. Learning data can further include negative examples that indicate when a desired result has not been achieved (e.g., when an estimate 228 is determined to be outside of a certain percentage or dollar amount of accuracy, when an estimate changes to an amount outside of a certain percentage or dollar amount of an elasticity estimate 230 that was previously determined by the estimator 210). According to an aspect, the learning data are used by the learning component of the estimator 210 to discover and generate new elasticity estimate rules and to measure the accuracy and effectiveness of the rules once they have been learned and implemented.

According to an aspect, a rule may be applied that instructs the actionator 214 to determine whether to notify a client of an estimate 228. In some examples, the estimate 228 may be an updated estimate. The determination may be based on an evaluation of one or more business rules, which are stored in the data store 114c, for determining whether one or more conditions are satisfied for notifying the client. For example, the one or more business rules may evaluate whether a condition is satisfied that a contracted payer is linked to the estimate 228. As another example, the one or more business rules may evaluate whether a condition is satisfied that the estimate 228 value is not $0. As another example, the one or more business rules may evaluate whether the estimate 228 is an updated estimated (the estimate has changed from a previous estimate determined by the estimator 210 and stored in the data store 114c). As another example, the one or more business rules may evaluate whether the client has opted into receiving estimate updates. The one or more business rules may further evaluate whether the difference between the amount of the previously-determined estimate and the updated estimate 228 satisfies a minimum threshold value for notifying the client.

When a determination is made to notify the client, the actionator 214 is further configured to orchestrate a combination or compilation of various data for submitting to the client navigation system 118 for notifying the client. The various data can include, but are not limited to, the normalized message 202 data, eligibility verification results data, the estimate 228, and/or the elasticity estimate 230.

For example, when the data are received by the client navigation system 118, a rule may be applied that instructs a notification engine 216 to notify the associated client of a determined estimate 228. The notification engine 216 is operative or configured to generate and transmit a notification 226 to the client that informs the client that an estimate 228 or an updated estimate is available. In various implementations, the notification engine 216 is embodied as a text messaging system, and the notification 226 is embodied as a text message transmitted to a client computing device 102 (e.g., mobile phone) associated with a mobile phone number in the client's record or provided by the client in association with consenting to receiving estimate notifications. The client computing device 102 (e.g., mobile phone) may comprise a text messaging application, which is configured to receive text messages from senders, including the notification engine 216 of the pre-service optimization system 122.

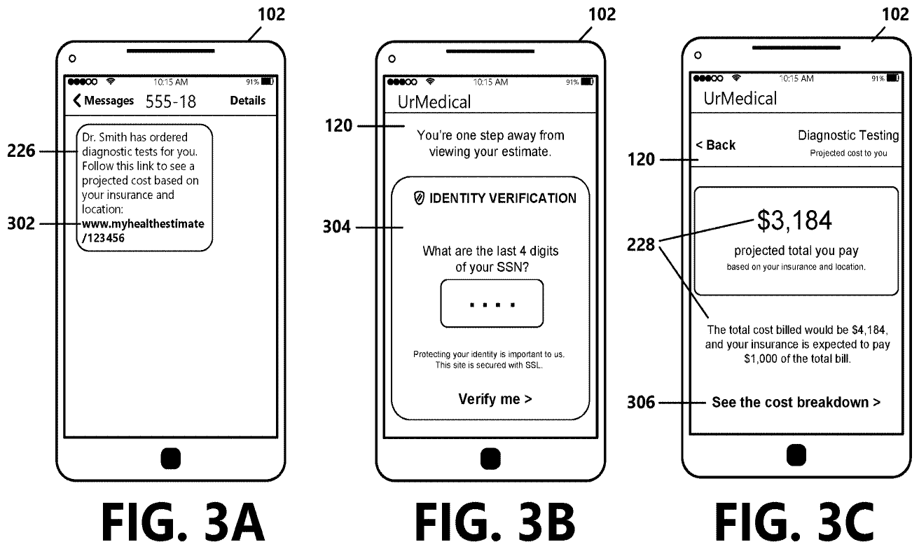

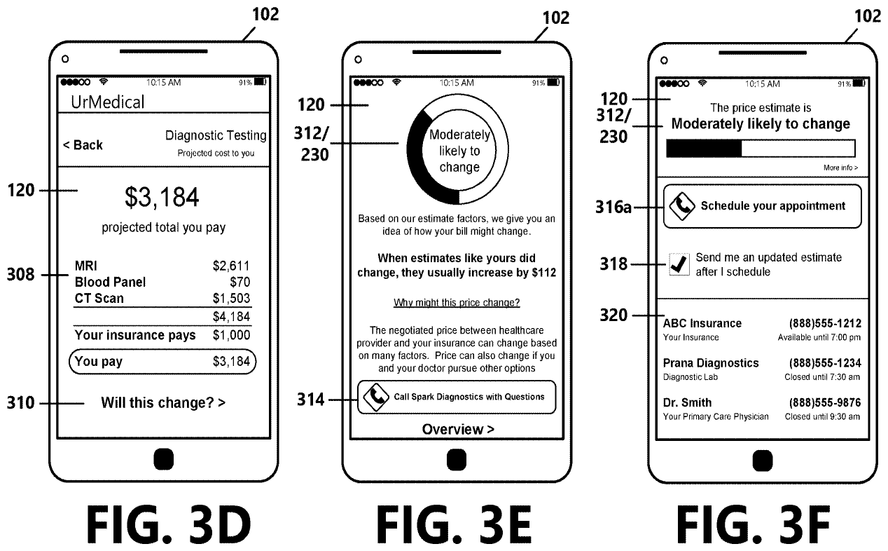

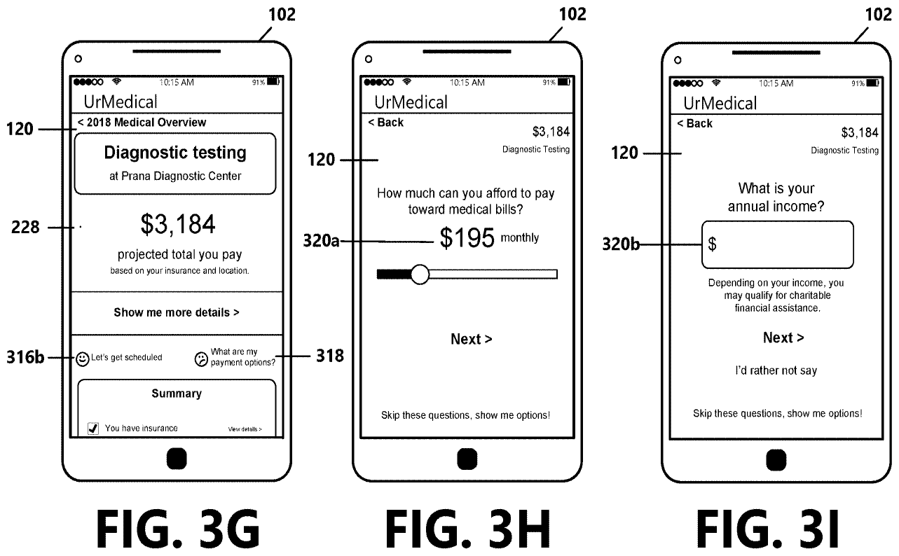

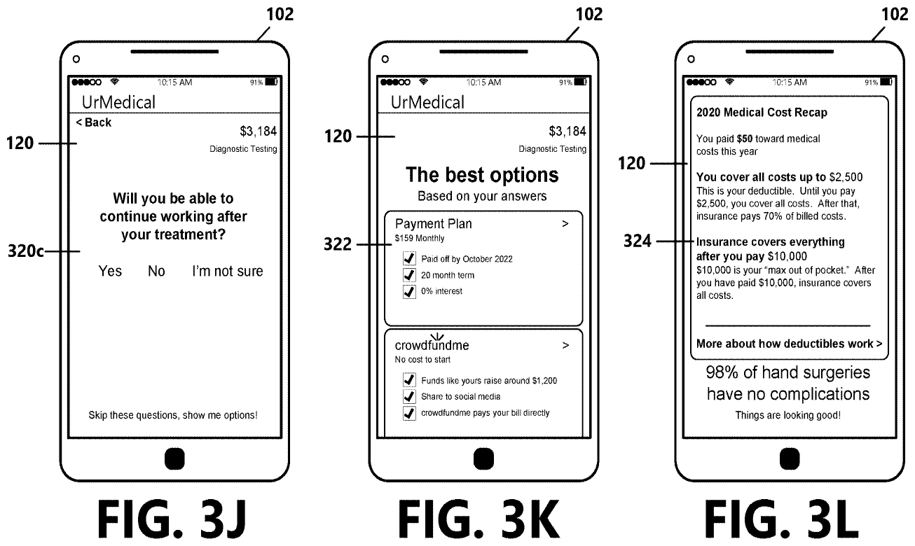

In other implementations, the notification engine 216 may be embodied as a push notification application or system configured to generate and push notifications 226 embodied as push notifications to a client application installed on the client computing device 102. For example, the client application may be configured to communicate with the notification engine 216, wherein the client application may receive push notifications from the notification engine 216 for display to the client and/or to generate local notifications based on a notification trigger received from the notification engine 216. For example, the notification trigger received from the notification engine 216 may include a notification that an estimate 228 has been generated and is available for the client to access. According to an aspect, the notification 226 (e.g., text message, push notification, notification trigger) may be pushed to the client when an estimate 228 is available and/or when an estimate has changed by a predetermined minimum amount (e.g., a certain dollar amount or percentage). As an example, an estimate 228 may change as new or updated information is received from a service provider or payer, such as updates to a treatment plan, procedures that may be performed, equipment that may be used, place where the service is to be performed, changes to the client's deductible amounts, etc. Accordingly, timely and accurate data may be pushed to the client pre-service, which enables the client to finalize financial readiness prior to the service. An example notification 226 embodied as a text message delivered to a client's mobile phone (client computing device 102) is illustrated in FIG. 3A. Other notification types are possible and are within the scope of the present disclosure.

According to an aspect, the notification 226 may include a selectable link, which when selected, directs the client computing device 102 to the client navigation system 118, which includes a user interface (UI) engine 220 configured to generate the client navigation UI 120 for display on the client's computing device. As will be described in further detail below, the client navigation UI 120 provides authenticated clients with access to service-related information related to the client.

In various implementations, selection of the link may direct the client computing device 102 to an authentication engine 218 associated with the client navigation system 118. The authentication engine 218 is illustrative of a software application, module, or computing device operative or configured to authenticate clients using a suitable authentication technology for allowing access to secure client-related content. For example, the authentication engine 218 may require the user to input identifying information to prove that the client is who he/she says that he/she is and to determine what information the client is authorized to access. The authentication engine 218 may be configured to verify the identifying information provided by the client against identifying information stored in the data store 114c for verifying the client's identity. When the user's identity is successfully authenticated, the access control engine may be further configured to request the client's access privileges against an authorization policy or set of permissions stored in the data store 114c for determining what information the client is authorized to access. The authentication engine 218 may be a single system that is configured to perform authentication and authorization processes; or, the authentication engine 218 may include separate authentication and authorization engines, wherein the authentication engine is configured to perform authentication processes and the authorization engine is configured to perform authorization processes.

The client navigation system 118 is illustrative of a secure web-based platform that can be accessed by a user agent (e.g., a web browser or a client application) executing on the client computing device 102. According to an aspect, the client navigation system 118 provides an authenticated client with access, via the client navigation UI 120, to information related to a service or services that have been ordered or scheduled for the client. For example, the client can use the client navigation UI 120 for one or a combination of the following activities: viewing an estimate 228 and/or an elasticity estimate 230 determined by the estimator 210 for an ordered or scheduled service or services, accessing a financial assistance screening interface provided by a financial assistance engine 212 for enabling the client to determine whether he/she may be eligible to receive financial assistance, access a payment interface provided by a payment system 224 for enabling the client to pre-pay or set up a payment plan for the ordered or scheduled service(s), and in some examples, accessing a scheduling interface provided by a scheduling system 222 for enabling the client to schedule ordered service(s).

According to an aspect, the elasticity estimate 230 can be provided to a client with the estimate 228 for informing the client of how likely the estimated amount that the client will owe for the ordered or scheduled service(s) may change, an amount that the estimate 228 is likely to change by, and factors or trigger points on which the elasticity estimate 230 is based (e.g., factors that may cause the estimate 228 to vary). For example, if the client is seeking treatment for a broken bone, the estimate 228 may be based on a treatment by a particular service provider involving a traditional plaster cast for the broken bone, and further based on the client's eligibility to receive benefits (e.g., payment of at least a portion of the treatment costs, discount to the treatment costs) from the client's payer. The corresponding elasticity estimate 230 may include a probability value that indicates the likelihood or chance that the estimate 228 may change. For example, from historical data, the estimator 210 may learn that a calculated percentage of the time, the estimated value and the actual costs billed to clients for treatment of the particular bone in view of the particular service provider and in view of the client's third-party payer differ in amount. The estimator 210 may further learn, based on historical data, that when the estimate 228 amount and the client responsibility amount differ, they typically differ within a certain dollar range. This may be due at least in part to a factor that an alternative course of treatment (e.g., a water-proof fiberglass cast) may be selected for treatment of the broken bone, which affects the costs of the treatment, and may additionally affect the amounts for which the user's payer may be responsible. The elasticity estimate 230 may be calculated based on this learned information. As part of providing the elasticity estimate 230 to the client via the client navigation UI 120, the elasticity estimate may include an explanation or details about the factors that may cause the actual client responsibility amount to vary from the estimate 228 value.

The financial assistance engine 212 is illustrative of a software application, module, or computing device operative or configured to make a determination as to whether or not the client may qualify for charity or financial assistance for the service(s) included in a received message 202. In some examples, the client navigation UI 120 may include a set of questions that request financial information from the client, which may correspond to the client's income and household size. The client's responses to these questions may be transmitted to the financial assistance engine 212 for making the determination. In other examples, a selectable functionality may be provided in the client navigation UI 120, which when selected, applies a rule for the UI engine 220 to communicate with the financial assistance engine 212. The financial assistance engine 212 may be configured to provide a set of questions to the client, which guides and enables the client to self-initiate a financial assistance qualification screening before services are rendered.

According to an aspect, in providing the interface for the financial assistance engine 212 in the client navigation UI 120 and enabling the client to self-initiate the financial assistance qualification screening before services are rendered, service provider processing resources can be utilized more efficiently. For example, the utilization of processing resources corresponding to billing statements and communications for recouping amounts owed for services that the client may not be able to afford can be reduced. In an example aspect, the financial assistance engine 212 may request financial information from the client corresponding to the client's income and household size. The financial assistance engine 212 may receive user responses and compare the received responses against an income threshold or other qualifying criteria to determine eligibility for charity care programs offered by the government, private charities, or the healthcare provider itself.

For example, a variety of assistance programs may be available to cover all or a portion of a client's responsibility amount for services provided by a service provider. These assistance programs can include assistance programs provided by the service provider and/or assistance programs provided by third-party organizations, such as private companies, charitable organizations, and government agencies. Financial assistance provided by an assistance program may be offered to clients who qualify for the program. Guidelines and criteria for qualification for an assistance program may be defined by rules stored in the data store 114c. For example, the rules may define types of services that are eligible for assistance, participating providers (e.g., physicians), qualifiers associated with a user's financial situation, insurance coverage/eligibility, or demographic information, and other service provider-specific assistance guidelines.

According to an aspect, when a determination is made that the client qualifies for charity care or financial assistance, the financial assistance engine 212 may be configured to communicate the determination/outcome to the client navigation system 118 for updating the client navigation UI 120 for notifying the client of the determination. In an example aspect, the financial assistance engine 212 is configured to communicate the determination/outcome to the BEP 206, which records the outcome in the data store 114c. In some implementations, a message is sent to the service provider system 104, which may notify an administrative user to contact the client about financial assistance or charity care programs for which the client qualifies.

The scheduling system 222 is illustrative of a software application, module, or computing device configured to provide scheduling functionalities for one or more healthcare providers (e.g., scheduling of appointments, procedures, and services). According to one example, the scheduling system 222 is part of the service provider system 104. According to another example, the scheduling system 222 is part of a third-party resource 106 that provides scheduling services to service providers. In some implementations, the scheduling system 222 exposes an application programming interface (API) configured to enable other systems, such as the client navigation system 118, to interface with the scheduling system 222. For example, the programmatic interface may be configured to interface the scheduling system 222 for enabling a client to schedule an ordered service or to cancel a scheduled service with the service provider through the client navigation UI 120.

The payment system 224 is illustrative of a software application, module, or computing device operative or configured to provide payment processing functionalities, such as allowing payments of the estimate 228 amount to be processed through one or more payment platforms, such as credit card, check, money order, cash, etc. The payment system 224 may further provide payment plan options from which the client is enabled to select for payment of the estimate 228 amount. The payment system 224 may be part of the service provider system 104, or may be part of a third-party resource 106 that provides payment services to service providers. In some implementations, the payment system 224 is configured to expose an API via which the client navigation system 118 can interface the payment system. For example, the client may remit payment for an ordered or scheduled service according to the client responsibility amount included in the estimate 228 or set up a payment plan to pay the client responsibility amount over a period of time.

The client navigation system 118 may be configured to interface with other systems for providing additional pre-service functionalities via the client navigation UI 120. In some examples, the client navigation system 118 may interface one or more of: a foreign alerts system and an associated registration quality assurance (RQA/PR) system configured to determine the quality of a registration to make sure all the data has been entered properly, to ensure entered data matches data provided by a payer, a credit reporting agency, or other third-party system for a client; an address verification system configured to verify a client's address for verifying received address information against other data (e.g., external data sources) available for the patient for determining whether the client lives where he/she says he/she lives; a medical necessity system that is configured to determine whether a government payment source (e.g., MEDICARE) will cover costs for a particular service for a particular client; an authorization or pre-certification system configured to interact with payers to determine authorization for a requested service (e.g., some services or procedures require authorization, and the payer determines whether or not they will cover the service or procedure before the service is rendered); a client-facing system that is configured to allow a viewing and paying of bills for received services; and/or a coverage discovery system configured to determine whether a self-paying client has access to payment from one or more other sources. Other example systems functionalities that may be provided via the client navigation UI 120 are possible and are within the scope of the present disclosure. According to aspects, the client navigation UI 120 navigates the client through the pre-service workflow, which includes steps to finalize financial readiness prior to the service (e.g., viewing an estimate, pre-paying for an upcoming service, setting up a payment plan for an upcoming service, determining whether the client qualifies for financial assistance). Client navigation system UI 120 examples are illustrated in FIGS. 3B-L and are described below.

According to an aspect, information provided to a client in an estimate 228 and/or an elasticity estimate 230 may help the client to make an informed decision regarding the scheduling of an associated service, which can reduce processing and memory resources used for scheduling and collections. For example, when the client is provided with estimate and change estimate information, the client may choose to schedule or choose not to schedule a service based on this information. Without the estimate and change estimate information, the client may not have the information needed to make an informed decision, and may schedule a service and then later cancel at the time of service when a more accurate estimate may be known (e.g., CPT codes and benefit data may be updated, which can change the estimate). As can be appreciated, this can negatively impact the service provider. For example, if a service is scheduled and missed (e.g., cancelled at or near the time of service), the service provider is likely to lose money on the missed/cancelled appointment. Additionally, providing the client with estimate and change estimate information can help to prevent certain services from being scheduled and rendered that the client may have not elected to schedule if the cost for those services had been known pre-service. For example, the client may not be able to afford the service, and additional billing system resources may be utilized to attempt to collect payment for those services for which the client may not be able to pay.

With reference now to FIG. 3A, an illustration is provided of an example notification 226 pushed to the client to inform the client that an estimate 228 has been determined for an ordered or scheduled service and is available for the client to access. For example, the notification 226 is embodied as a text message pushed to the client's mobile phone (client computing device 102). As described above, the notification 226 may be pushed to the client computing device 102 when an estimate 228 is available and/or when a previously determined estimate has changed by a predetermined threshold amount (e.g., a certain dollar amount or percentage). The example notification 226 includes a link 302, which when selected, may direct the client computing device 102 to the authentication engine 218 of the client navigation system 118 for enabling the client to authenticate him/herself for allowing the client access to service-related information related to the client. Accordingly, timely and accurate data may be pushed to the client pre-service, which enables the client to finalize financial readiness prior to the service.

As illustrated in FIG. 3B, the authentication engine 218 is configured to provide an authentication UI 304 for enabling the client to provide credentials for enabling the authentication engine 218 to verify the client's identity for allowing access to the estimate 228. When the client's identity is verified, the UI engine 220 may update the client navigation UI 120 to include a display of the estimate 228. As an example and with reference to FIG. 3C, an illustration of an example estimate 228 displayed in an example client navigation UI 120 is provided.

In some examples, the client navigation UI 120 is provided as a webpage generated by the UI engine 220 of the client navigation system 118 that the client accesses via a web browser or other user agent on the computing device 102. In other examples, the client navigation UI 120 is generated by a dedicated application executing on the client's device 102 that is configured to communicate with the client navigation system 118 to receive estimate 228 information for displaying to the client. The client navigation UI 120 may be configured to receive data entered by the client for communicating with the client navigation system 118 and to receive information from the client navigation system for providing to the user (e.g., client, guarantor) of the client navigation UI 120.

As illustrated in FIG. 3C, the example client navigation UI 120 includes an estimate 228 for an ordered or scheduled service or services in association with a received message 202. The estimate 228 can include the client responsibility amount for the service(s), and may additionally include a total amount that is expected to be billed and an amount expected to be paid by a third-party payer (e.g., the client's insurance company). For example, the client responsibility amount can be based on the projected total costs for the service(s), less the amount expected to be paid by the third-party payer based on eligibility verification information, such as a deductible amount, co-pay amount, etc.

In some examples, the client navigation UI 120 may include other information associated with the estimate 228, such as a listing of factors used to determine the estimate (e.g., the client's insurance and the client's location). As can be appreciated, this listing of factors can be a high-level summary of factors, and may not include all factors evaluated for determining the estimate 228. In various implementations, a selectable cost breakdown link 306 may be provided that, when selected, updates the UI 120 to include a breakdown of the estimate 228.

With reference to FIG. 3D, an example illustration of the client navigation UI 120 is provided wherein the UI is updated to display the cost breakdown 308 of the estimate 228. For example, the cost breakdown 308 can include a listing of the cost(s) of the one or more services included in the estimate 228, discounts applied to the service costs, and/or an amount expected to be paid by the client's insurance/third-party payer. In some implementations, the UI 120 includes a selectable link 310, that when selected, updates the UI 144 to include a display of a determined elasticity estimate 230.

With reference now to FIG. 3E, an example illustration of the client navigation UI 120 is provided wherein the UI is updated to display an indication 218 of the determined elasticity estimate 230. For example this indication 312 can be embodied as text, a graph, or other visual or audible representation of the determined likelihood that the calculated estimate 228 will change. In various implementations, the elasticity estimate 230 displayed in the UI 120 includes an amount determined by the estimator 210 as an expected amount by which the estimate 228 may change. In various implementations, the elasticity estimate 230 includes a listing of factors that can cause the estimate 228 to change. In various implementations, one or more selectable UI controls 314 are provided, which when selected, enable the client to selectively contact (e.g., call, message, chat with) the service provider. For example, selection of a selectable UI control 314 can cause a messaging application on the client's computing device 102 to open and to populate a recipient field with a messaging contact number for the service provider, cause a phone application to open and to populate a recipient field with a phone number for the service provider, cause an email application to open and to populate a recipient field with an email address for the service provider, etc.

With reference to FIG. 3F, another example illustration of the UI 120, wherein the UI is updated to display an indication 312 of the determined elasticity estimate 230. In the illustrated example, the indication 312 is embodied as a different type of visual representation than illustrated in FIG. 3E. As should be appreciated, various types of indicators are possible and are within the scope of the present disclosure. In various implementations, the UI 120 includes a selectable UI control 316, which when selected enables the client navigation system 118 to interface the scheduling system 222. For example, responsive to a selection of the selectable UI control 316a, the UI 120 may be updated to include an interface for the scheduling system 222, which enables the client to schedule the service(s) associated with the estimate 228 or to cancel a prior-scheduled service(s) associated with the estimate.

In various implementations, the client navigation UI 120 includes another selectable UI control 318, which when selected, opts the patient into receiving updated estimates. For example, selection of the other selectable UI control 318 may update a business rule in the client's client profile stored in the data store 114c that instructs the actionator 214 that the client has opted into receiving estimate updates, and thus to make a determination to notify the client of an updated estimate 228 when an estimate is updated.

In various implementations, the client navigation UI 120 includes one or more contact data 320, wherein the contact data can include a listing of one or more service providers associated with providing the service(s) (e.g., a healthcare provider associated with ordering the service, a healthcare provider associated with providing the service), contact information (e.g., phone numbers, website links, email addresses, street addresses) of the one or more healthcare providers, hours of operation of the one or more healthcare providers, etc.

With reference to FIG. 3G, an example illustration of the client navigation UI 120 is provided that includes another example of a selectable UI control 316b, which when selected enables the client navigation system 118 to interface the scheduling system 222. In various implementations, the UI includes another selectable UI control 318, which when selected, causes the client navigation system 118 to update the UI 120 to include a display of payment options.

With reference to FIG. 3H, an example illustration of the client navigation UI 120 is provided, wherein the UI is updated to display a request for financial information input from the client that can be used to determine eligibility for charity care or financial aid programs offered by the government, private charities, or the healthcare provider itself for the benefit of the public. For example, a first example UI input element 320a is illustrated that can be provided in the client navigation UI 120 for enabling the client to input or indicate an amount that the client can afford to pay each month toward payment for the service(s). With reference to FIG. 3I, a second example UI input element 320b is illustrated that can be provided in the client navigation UI 120 for enabling the patient to input or indicate the patient's annual income. With reference to FIG. 3J, a third example UI input element 320c is illustrated that can be provided in the client navigation UI 120 for enabling the client to input or indicate whether the client will be able to continue to work after receiving the service(s). As should be appreciated, the above example UI input elements 320 are not meant to limiting, but are examples of some of various UI input elements that can be provided in the client navigation UI 120 for receiving various financial information from the patient for determining the client's eligibility for charity care programs. Other UI input element types can be provided and other financial information can be collected and are within the scope of the present disclosure.

In some examples, an interface for the financial assistance system 212 is provided in the client navigation UI 120 via an API. The client input may be transmitted to the financial assistance screen system 212, where a determination may be made as to whether the patient is eligible for a charity care program or for other financial assistance. A result of the financial assistance screen system determination 212 may be communicated to the client navigation system 118, and the UI engine 220 may be configured to update the client navigation UI 120 to display the results. According to an aspect, when a determination is made that the patient is eligible for financial assistance, the UI 120 may be updated to include the positive determination result. In some implementations, the client navigation UI 120 may additionally include a link to access a charity care or financial assistance program resource (e.g., third party resource 106). According to another aspect and with reference to FIG. 3K, the client navigation UI 120 may be updated to include one or more payment options 322, such as a payment plan (e.g., based financial information input by the patient), a crowd-sourced payment option, etc.

With reference to FIG. 3L, an example illustration of the client navigation UI 120 is provided, wherein the UI is updated to display a coverage summary 324 for the client. According to an aspect, eligibility verification information provided by a payer to the eligibility verifier 208 may be summarized and included in the client navigation UI 120. For example, the coverage summary 324 can include one or more of: an amount that the client has paid towards medical costs for the current year, a deductible amount that the client is responsible to pay out-of-pocket before the payer covers all or a portion of additional costs, an out-of-pocket maximum amount, and a link to additional information about the client's benefits and coverages, etc. For example, the link may direct the client to general information about insurance coverages, or may direct the client to the client's payer (e.g., third party resource 106).

In an example aspect, eligibility data provided by a payer to the eligibility verifier 208 can indicate that the client's remaining deductible amount is $0 or may be $0 after the service(s) are provided to the client. Accordingly, in some implementations, based on this information, the pre-service optimization system 122 may further include a recommendation engine configured to determine one or more other services to recommend to the client as services that may be relevant to the client. For example, the client may be unaware that his/her deductible has been met, and may not realize that he/she can advantageously select and schedule one or more additional services (that may or may not be related to the original service associated with the estimate 228), the costs for which may be covered up to a certain percentage or completely by the payer. In some implementations, the one or more services recommended to the client may be included based on a list of recommended services provided by the service provider system 104 and stored in the data store 114c. In other implementations, the one or more services recommended to the client can be included based on an evaluation of rules stored in the data store 114c for determining recommended services for the client based, for example, on the patient's age, sex, medical history, etc. Upon selection of a service, the client navigation system 118 may be configured to interface the scheduling system 222 for scheduling the selected service, or otherwise initiating a workflow for scheduling the service or scheduling an appointment with the service provider for learning more about the selected service. As can be appreciated, more or less information may be provided in the client navigation system UI 120.

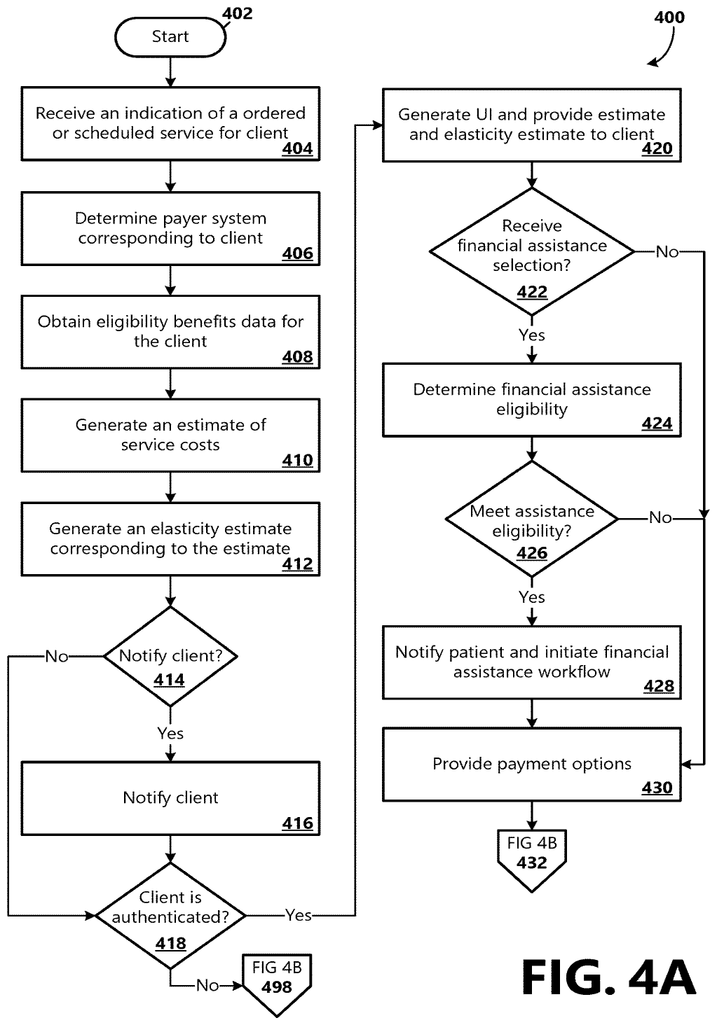

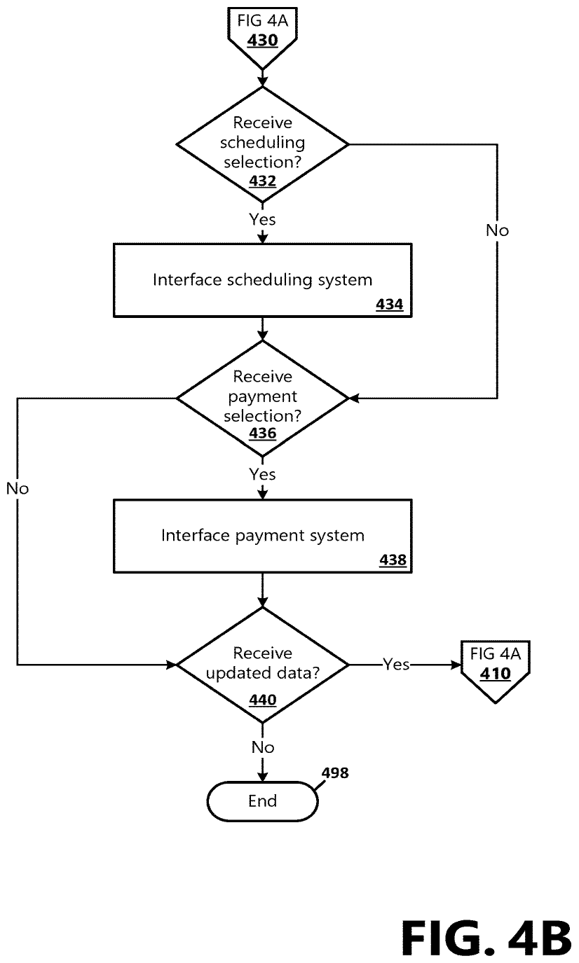

FIGS. 4A-B illustrate a flow chart showing general stages involved in an example method 400 for providing pre-service workflow optimization. Method 400 begins at START OPERATION 402 and proceeds to OPERATION 404, where the method 400 uses the message processor 203 to receive an indication of an ordered or scheduled service(s) for a client. In example aspects, the indication of the ordered or scheduled service(s) is a message 202 sent from a service provider system 104 to the pre-service optimization system 122 that includes information about one or more service(s) ordered or scheduled for the client, an identification of the client, and other information. At OPERATION 404, the method 400 may further use the message processor 203 to normalize and evaluate the message 202 for determining whether one or more criteria are met for generating an estimate 228 for the one or more services, wherein the estimate includes an estimated amount for which the client is responsible. As described above, in example aspects, the one or more criteria may be associated with the type of service ordered or scheduled for the client, the payer (e.g., commercial payer or self-pay versus a government assistance based program), or other factors. The criteria may be configurable and defined by each service provider. Responsive to determining to run an estimate for the received message 202, the method 400 may further utilize the message processor 203 to route the normalized message 202 to the FEP 204.

At OPERATION 406, the method 400 may use the FEP 204 to create an event, which operates as a trigger to one or more pre-service optimization system 122 components to perform certain operations for determining an estimate 228 and/or an elasticity estimate 230 for a service or services associated with the received message 202. Additionally, at OPERATION 406, the method 400 may further use the FEP 204 to perform an encounter matching process for accessing the client's insurance coverage information stored in the data store 114c for determining whether there is a payer (e.g., private insurance provider, governmental insurance provider, pharmaceutical company) on record in association with the client and if so, whether the pre-service optimization system 122 has connectivity to the payer. In an example aspect, the patient's insurance coverage information may include insurance data entered when onboarding the client in the service provider system 104 or another service provider system. For example, insurance data can be on record in association with the client if the client has a client account established with the service provider or another service provider that is in communication with the pre-service optimization system 122 and that is authorized to share data with the pre-service optimization system. The insurance data can be used to identify an insurance provider (if any), any available secondary insurers (e.g., Medicare), and particular plans to which the client (or guarantor) may be subscribed.

When a payer is identified for the client and is a payer that a system has connectivity to, at OPERATION 408, the method 400 may use the eligibility verifier 208 to generate and transmit an eligibility verification inquiry or request to the identified payer (e.g., third party resource 106) for determining the client's eligibility for insurance coverage and benefits. In response to the inquiry or request, the payer may transmit an eligibility verification response that includes details of the client's coverage, benefits, and eligibility such as the client's benefit status, explanation of benefits, coverages, effective dates, amounts for co-insurance, co-pays, deductibles, exclusions, and limitations. In some example aspects, the method 400 further utilizes the eligibility verifier 208 to apply eligibility rules for determining whether the client's eligibility is valid. In some example aspects, the method 400 may further utilize the eligibility verifier 208 to apply conversion rules to convert raw eligibility data provided by the payer system into actionable information that can be used by the estimator 210 for generating an estimate 228 and/or an elasticity estimate 230. According to an aspect, the method 400 uses the eligibility verifier 208 to communicate eligibility benefits data to the BEP 206, wherein the method 400 may utilize the BEP 206 to store the eligibility benefits data in the data store 114c, compile the normalized message data and the eligibility benefits data, and pass the compiled data to the estimator 210.

At OPERATION 410, the method 400 uses the estimator 210 to generate an estimate 228 for the one or more services associated with the received message 202. For example, as part of generating the estimate 228, the estimator 210 may use the eligibility benefits data, the normalized message data, and service information corresponding to the service provider. In example aspects, the service information may include costs associated with the one or more services, as set by the healthcare provider or as negotiated between the healthcare provider and the client's payer. In various aspects, the eligibility benefits data obtained at OPERATION 410 may include pricing information agreed to by the service provider and the payer. For example, the pricing information may specify prices that the payer and the service provider have negotiated for various services or diagnoses. In other aspects, service information are stored in the data store 114c and are accessed by the estimator 210 for generating the estimate. For example, the estimator 210 may generate an estimate 228 by determining which services may be performed, which equipment and supplies may be used, and the costs associated with these services, equipment, and supplies (e.g., based on a contract between the service provider and the payer). The estimator 210 may further generate the estimate 228 based on the client's eligibility, copay amounts, and deductible amounts. In some examples, when multiple services are part of a course of treatment, their associated costs may be added together for processing as one transaction. In other examples, each service may be processed separately, for example, an ambulance ride and a subsequent emergency room visit may be estimated and/or billed separately or together. After generating the estimate 228, the estimate may be stored in the data store 114c.

At OPERATION 412, the method 400 utilizes the estimator 210 to generate an elasticity estimate 230 for the estimate 228 generated at OPERATION 410. For example, the method 400 uses a model trained by a machine-learning algorithm of the estimator 210 to determine a likelihood that the estimate 228 will change and a range or an amount that the estimate is likely to change by based on learning data (e.g., training data and historical data). For example, the machine-learning algorithm may be ran over the learning data to learn trigger points associated with changes to estimates 228 and effects of those trigger points on the estimates, wherein the trigger points are associated with various factors that affect the difference between one or more estimates 228 of a service or services that a client is seeking and the actual service(s) provided to the client and a final cost of the service(s) that is ultimately billed to the client or a guarantor of the client. As part of generating the elasticity estimate 230, the estimator 210 may use the model to analyze the eligibility benefits data, the normalized message data, and/or service information to calculate a probability that the estimate 228 will change and/or a dollar amount range within which the estimate is likely to change. After generating the elasticity estimate 230, the elasticity estimate may be stored in the data store 114c.

At DECISION OPERATION 414, the method 400 uses the actionator 214 to make a determination as to whether to notify the client of the estimate 228 and/or the elasticity estimate 230. For example, the determination may be based on an evaluation of one or more business rules stored in the data store 114c for determining whether one or more conditions are satisfied for notifying the client (e.g., whether a contracted payer is linked to the estimate 228, whether the estimate 228 value is non-$0, whether the estimate 228 is an updated estimated (the estimate has changed from a previous estimate determined by the estimator 210 and stored in the data store 114c); whether the difference between the amount of the previously-determined estimate and the updated estimate 228 satisfies a minimum threshold value; whether the client has opted into receiving estimate updates).

When the actionator 214 makes a determination to notify the client, at OPERATION 416, the method 400 may further use the actionator 214 to orchestrate a compilation of various data (e.g., the normalized message 202 data, eligibility verification results data, the estimate 228, and/or the elasticity estimate 230) for submitting to the client navigation system 118 for notifying the client. The method 400 may further utilize the client navigation system 118 to generate and transmit a notification 226 (e.g., text message, push notification) to the client's computing device 102 (e.g., mobile phone associated with a mobile phone number in the client's record or provided by the client in association with consenting to receiving estimate notifications) that informs the client that an estimate 228 or an updated estimate is available and provides a link for enabling the client to access the estimate 228 and the elasticity estimate 230. A text messaging application, service provider-associated client application, or the like operating on the client computing device 102 (e.g., mobile phone) may receive the notification 226 and display the notification 226 to the client. According to an aspect, the notification 226 is delivered to the client as data related to an upcoming service for the client is received and as the client's estimated client responsibility amount is determined. Accordingly, the client is informed of a revised financial responsibility in real time or near-real time, which provides the client with the maximum amount of time to react to the revised estimate 228 and to finalize financial readiness prior to the service.

At DECISION OPERATION 418, the method 400 uses the client navigation system 118 to determine whether the client's identity has been authenticated (e.g., by the authentication engine 218) for allowing the client to access the client navigation UI 120 for receiving the estimate 228 and the elasticity estimate 230. When the patient's identity is authenticated, at OPERATION 420, the method 400 uses the UI engine 220 to generate the client navigation UI 120 for displaying the estimate 228 and the elasticity estimate 230 and other relevant information. According to an aspect, the client navigation UI 120 may further include one or more interfaces to one or more other systems (e.g., scheduling system 222, payment system 224, financial assistance engine 212) for enabling the client to be guided through a pre-service workflow process and to finalize readiness for the services prior to the receiving the services.

At DECISION OPERATION 422, a determination may be made as to whether a client selection or input is received via the client navigation UI 120 to initiate a workflow for determining whether the patient meets criteria for receiving charity care or financial assistance from one or more charity care or financial assistance programs. Responsive to a client selection of a financial assistance option, at OPERATION 424, the method 400 may use the financial assistance screening engine 212 to determine the patient's eligibility for charity care or financial assistance for the upcoming service(s) associated with the estimate 228. In some examples, the financial assistance screening engine 212 may receive client input in response to one or more screening questions provided to the client in the client navigation UI 120, and evaluate whether the received client input meet criteria that may be set by the healthcare provider (e.g., for a pro bono treatment program), by third party programs that may provide criteria to the pre-service optimization system 122 (e.g., local, national, or international aid societies for various medical conditions), or by governmental aid programs.

At DECISION OPERATION 426, the method 400 may use the financial assistance screening engine 212 to make a determination as to whether the client meets assistance eligibility or not. When a determination is made that the client is eligible, at OPERATION 428, the method 400 may use the client navigation system 118 to notify the client. The method 400 may further use the client navigation system 118 to initiate a financial assistance workflow. For example, the client navigation system 118 may send a notification to an administrative user of the service provider system 104 that informs the service provider of the client's eligibility, documents may be provided to the client for completing a financial assistance registration/application process, or a link to a financial assistance program system that the patient qualifies for can be provided to the patient via the client navigation UI 120. At OPERATION 430, various payment options may be provided to the client via the client navigation UI 120, which enables the client to select a payment plan, a crowd-sourced payment option, or to access the payment system 224 (e.g., via a link or an API provided in the client navigation UI 120) for making a payment for the service(s) prior to the services being rendered.

With reference now to FIG. 4B, at DECISION OPERATION 432, the method 400 may utilize the client navigation UI 120 to determine whether a client-selection is made to schedule or cancel the service(s). For example, a user-selection can include a selection of a UI functionality, which when selected, enables the client navigation system 118 to interface with the scheduling system 222. For example, responsive to a selection of a scheduling UI control for the scheduling system 222, at OPERATION 434, the method 400 may use the UI engine 220 to direct the client to the scheduling system or to communicate with the scheduling system 222 (via an API). In some examples, functionalities of the scheduling system 222 may be provided via the client navigation UI 120, enabling the client to schedule the service(s) associated with the estimate 228 or to cancel or reschedule a prior-scheduled service or services associated with the estimate.

At DECISION OPERATION 436, the method 400 may utilize the client navigation UI 120 to determine whether a client-selection is made to make a payment. For example, a client-selection can include a selection of a selectable payment UI control. Responsive to a selection of the UI control, at OPERATION 438, the method 400 may use the UI engine 220 to direct the client to the payment system 224 or to communicate with the payment system 224 (via an API). For example, the client may interact with the payment system 224 to pay at least a portion of the client responsibility amount for the upcoming service(s) associated with the estimate 228.

At DECISION OPERATION 440, the method 400 may use the message processor 203 to receive a second message from the service provider system 104, wherein the second message may include an update to an upcoming service for which the pre-service optimization system 122 has previously generated an estimate 228. For example, the method 400 may further use the message processor 203 to normalize and evaluate the second message for determining whether the second message includes updated data (e.g., updated CPT codes, benefits data) associated with an upcoming service associated with a stored estimate 228. When a determination is made that the second message includes updated data for an upcoming service, the method 400 may return to OPERATION 410, where the method 400 uses the estimator 210 to generate an updated estimate 228 and an updated elasticity estimate 230 for the one or more services associated with the updated data received in the second message. The method 400 may proceed through the subsequent OPERATIONS until the method ends at OPERATION 498.

FIG. 5 is a block diagram illustrating physical components of an example computing device with which aspects may be practiced. The computing device 500 may include at least one processing unit 502 and a system memory 504. The system memory 504 may comprise, but is not limited to, volatile (e.g. random access memory (RAM)), non-volatile (e.g. read-only memory (ROM)), flash memory, or any combination thereof. System memory 504 may include operating system 506, one or more program instructions 508, and may include sufficient computer-executable instructions for a pre-service optimization system 122, which when executed, perform functionalities as described herein. Operating system 506, for example, may be suitable for controlling the operation of computing device 500. Furthermore, aspects may be practiced in conjunction with a graphics library, other operating systems, or any other application program and is not limited to any particular application or system. This basic configuration is illustrated by those components within a dashed line 510. Computing device 500 may also include one or more input device(s) 512 (keyboard, mouse, pen, touch input device, etc.) and one or more output device(s) 514 (e.g., display, speakers, a printer, etc.).

The computing device 500 may also include additional data storage devices (removable or non-removable) such as, for example, magnetic disks, optical disks, or tape. Such additional storage is illustrated by a removable storage 516 and a non-removable storage 518. Computing device 500 may also contain a communication connection 520 that may allow computing device 500 to communicate with other computing devices 522, such as over a network in a distributed computing environment, for example, an intranet or the Internet. Communication connection 520 is one example of a communication medium, via which computer-readable transmission media (i.e., signals) may be propagated.

Programming modules may include routines, programs, components, data structures, and other types of structures that may perform particular tasks or that may implement particular abstract data types. Moreover, aspects may be practiced with other computer system configurations, including hand-held devices, multiprocessor systems, microprocessor-based or programmable user electronics, minicomputers, mainframe computers, and the like. Aspects may also be practiced in distributed computing environments where tasks are performed by remote processing devices that are linked through a communications network. In a distributed computing environment, programming modules may be located in both local and remote memory storage devices.

Furthermore, aspects may be practiced in an electrical circuit comprising discrete electronic elements, packaged or integrated electronic chips containing logic gates, a circuit using a microprocessor, or on a single chip containing electronic elements or microprocessors (e.g., a system-on-a-chip (SoC)). Aspects may also be practiced using other technologies capable of performing logical operations such as, for example, AND, OR, and NOT, including, but not limited to, mechanical, optical, fluidic, and quantum technologies. In addition, aspects may be practiced within a general purpose computer or in any other circuits or systems.

Aspects may be implemented as a computer process (method), a computing system, or as an article of manufacture, such as a computer program product or computer-readable storage medium. The computer program product may be a computer storage medium readable by a computer system and encoding a computer program of instructions for executing a computer process. Accordingly, hardware or software (including firmware, resident software, micro-code, etc.) may provide aspects discussed herein. Aspects may take the form of a computer program product on a computer-usable or computer-readable storage medium having computer-usable or computer-readable program code embodied in the medium for use by, or in connection with, an instruction execution system.

Although aspects have been described as being associated with data stored in memory and other storage mediums, data can also be stored on or read from other types of computer-readable media, such as secondary storage devices, like hard disks, floppy disks, or a CD-ROM, or other forms of RAM or ROM. The term computer-readable storage medium refers only to devices and articles of manufacture that store data or computer-executable instructions readable by a computing device. The term computer-readable storage media do not include computer-readable transmission media.

Aspects of the present invention may be used in various distributed computing environments where tasks are performed by remote processing devices that are linked through a communications network.

Aspects of the invention may be implemented via local and remote computing and data storage systems. Such memory storage and processing units may be implemented in a computing device. Any suitable combination of hardware, software, or firmware may be used to implement the memory storage and processing unit. For example, the memory storage and processing unit may be implemented with computing device 500 or any other computing devices 522, in combination with computing device 500, wherein functionality may be brought together over a network in a distributed computing environment, for example, an intranet or the Internet, to perform the functions as described herein. The systems, devices, and processors described herein are provided as examples; however, other systems, devices, and processors may comprise the aforementioned memory storage and processing unit, consistent with the described aspects.

The description and illustration of one or more aspects provided in this application are intended to provide a thorough and complete disclosure the full scope of the subject matter to those skilled in the art and are not intended to limit or restrict the scope of the invention as claimed in any way. The aspects, examples, and details provided in this application are considered sufficient to convey possession and enable those skilled in the art to practice the best mode of the claimed invention. Descriptions of structures, resources, operations, and acts considered well-known to those skilled in the art may be brief or omitted to avoid obscuring lesser known or unique aspects of the subject matter of this application. The claimed invention should not be construed as being limited to any embodiment, aspects, example, or detail provided in this application unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently. Having been provided with the description and illustration of the present application, one skilled in the art may envision variations, modifications, and alternate embodiments falling within the spirit of the broader aspects of the general inventive concept provided in this application that do not depart from the broader scope of the present disclosure.

1.-20. (canceled)

21. A pre-service client navigation system for providing, comprising:
at least one processing device; and
at least one computer readable data storage device storing instructions that, when executed by the at least one processing device, cause the system to:
receive, by a message processor, a first message associated with a service provider system, wherein the first message includes message data specifying at least one upcoming service for a client, and wherein the message processor is configured to:
generate normalized message data by converting the message data from a first format to a second format, and
based on a determination that one or more criteria for generating an estimate are met, route the normalized message data to a front-end processor that is configured to:
receive the normalized message data in the second format,
match the normalized message data to a client record associated with the client, and
identify, within the client record, a payer system corresponding to the client;
obtain, by an eligibility verifier, eligibility benefits data for the client from the payer system, wherein the eligibility verifier is configured to:
generate and transmit, to the payer system, an eligibility verification request, and
receive, from the payer system, eligibility benefits data for the client;
input, into an estimator comprising a machine-learning model trained using first learning data, the eligibility benefits data, the normalized message data, and service information corresponding with the service provider system;
receive, from the estimator,
a first estimate of a client responsibility amount for the at least one upcoming service, and
a first elasticity estimate corresponding to the first estimate, wherein the first elasticity estimate comprises:
(1) a likelihood that the first estimate will change, and
(2) a numerical range within which the first estimate is likely to change;
generating and transmitting, by a client navigation system and to a computing device corresponding to the client, a notification comprising a link to access the first estimate and the first elasticity estimate;
receive, from the client computing device, an indication of a selection to access the client navigation system; and
generate display instructions configured to display a user interface including a pre-service workflow prior to the client receiving the at least one upcoming service, wherein the user interface includes the first estimate and the first elasticity estimate.

22. The system of claim 21, wherein the first message is received in response to the at least one upcoming service being ordered or scheduled for the client by the service provider system.

23. The system of claim 21, wherein the notification is embodied as at least one of:
a text message that can be received by a text messaging application operating on the client's computing device and displayed or played audibly to the client; or
a push notification that can be received by an application operating on the client's computing device or system and displayed or played audibly to the client.

24. The system of claim 21, wherein the system is further configured to:
receive updated information corresponding to the at least one service or to the eligibility benefits data;
determine a second estimate based on the updated information;
determine a second elasticity estimate associated with the second estimate; and
transmit a second notification to the client computing device, wherein the second notification includes a link to access the second estimate and the second change estimate.

25. The system of claim 21, wherein in generating the user interface, the system is configured to provide a unified portal that interfaces one or more workflow systems, wherein the one or more workflow systems include one or more of:
a scheduling system;
a payment system; and
a financial assistance screening system.

26. The system of claim 21, wherein the service information comprises costs associated with the at least one upcoming service.

27. The system of claim 21, wherein the first learning data comprises historical data comprising:
one or more services previously provided,
previously determined estimates for the one or more services,
final costs of providing the one or more services, and
the client's responsibility for the final costs.

28. The system of claim 21, wherein the eligibility benefits data includes pricing information agreed to by the service provider system and the payer system.

29. The system of claim 21, wherein the one or more criteria are configurable and defined by the payer system, and wherein the payer system is associated with a commercial payer, self-payer, or government assistance-based program.

30. The system of claim 21, wherein the payer system is associated with a commercial payer, self-payer, or government assistance-based program.

31. A method for providing pre-service client navigation, comprising:
receiving, by a message processor, a first message associated with service provider system, wherein the first message includes message data specifying at least one upcoming service for a client, and wherein the message processor is configured to:
generate normalized message data by converting the message data from a first message format to a second message format, and
based on a determination that one or more criteria for generating an estimate are met, route the normalized message data to a front-end processor that is configured to:
receive and process the normalized message data in the second message format,
match the normalized data to a client record associated with the client, and
identify, within the client record, a payer system corresponding to the client;
obtaining, by an eligibility verifier, eligibility benefits data for the client from the payer system, wherein the eligibility verifier is configured to:
generate and transmit, to the payer system, an eligibility verification request to the payer system, and
receive, from the payer system, eligibility benefits data for the client from the payer system;
inputting, into an estimator comprising a machine-learning model trained using first learning data, the eligibility benefits data, the normalized message data, and service information corresponding to the service provider system;
receiving, from the estimator,
a first estimate of a client responsibility amount for the at least one upcoming service based on, and
a first elasticity estimate corresponding to the first estimate, the first elasticity estimate comprising (1) a likelihood that the first estimate will change and (2) a numerical range within which the first estimate is likely to change;
generating and transmitting, by a client navigation system and to a computing device corresponding to the client, a notification comprising a link to access the first estimate and the first elasticity estimate;
receiving, from the client's computing device, an indication of a selection to access the client navigation system; and
generating, by the client navigation system, display instructions configured to display a user interface including a pre-service workflow prior to the client receiving the at least one upcoming service, wherein the user interface includes the first estimate and the first elasticity estimate.

32. The method of claim 31, wherein the first message is received in response to the at least one upcoming service being ordered or scheduled for the client by the service provider system.

33. The method of claim 31, wherein transmitting a notification to a computing device of the client comprises at least one of:
transmitting a text message that can be received by a text messaging application operating on the client's computing device and displayed or played audibly to the client; and
transmitting a push notification that can be received by an application operating on the client's computing device or system and displayed or played audibly to the client.

34. The method of claim 31, further comprising:
receiving, via the message processor, updated information corresponding to the at least one service or to the eligibility benefits data;
determining a second estimate based on the updated information;
determining a second elasticity estimate associated with the second estimate; and
transmitting, via the client navigation system, a second notification to the client computing device, wherein the second notification includes a link to access the second estimate and the second change estimate.

35. The method of claim 31, wherein generating the user interface comprises including, in the user interface, one or more interfaces to one or more workflow systems, the one or more workflow systems including one or more of:
a scheduling system;
a payment system; and
a financial assistance screening system.

36. The method of claim 31, wherein the service information comprises costs associated with the at least one upcoming service.

37. The method of claim 31, wherein the first learning data comprises historical data comprising:
one or more services previously provided,
previously determined estimates for the one or more services,
final costs of providing the one or more services, and
the client's responsibility for the final costs.

38. The method of claim 31, wherein the eligibility benefits data includes pricing information agreed to by the service provider system and the payer system.

39. A computer-readable storage device including computer readable instructions, which when executed by a processing unit are configured to:
receive, by a message processor, message associated with a service provider system,
wherein the message includes message data specifying at least one upcoming service for a client, and
wherein the message processor is configured to:
generate normalized message data by converting the message data from a first message format to a second message format, and
based on a determination that one or more criteria for generating an estimate are met, route the normalized message data to a front-end processor that is configured to:
receive and process the normalized message data in the second message format,
match the normalized data to a client record associated with the client, and
identify, within the client record, a payer system corresponding to the client;
obtain, by an eligibility verifier, eligibility benefits data for the client from the payer system, wherein the eligibility verifier is configured to:
generate and transmit, to the payer system, an eligibility verification request to the payer system, and
receive, from the payer system, eligibility benefits data for the client from the payer system;
input, into an estimator comprising a machine-learning model trained for using first learning data, the eligibility benefits data, the normalized message data, and service information corresponding to the service provider system;
receive, from the estimator,
a first estimate of a client responsibility amount for the least one upcoming service based on;
a first elasticity estimate corresponding to the first estimate, the first elasticity estimate comprising (a) a likelihood that the first estimate will change and (2) a numerical range within which the first estimate is likely to change;
generating and transmitting, by a client navigation system and to a computing device corresponding to the client, a notification comprising a link to access the first estimate and the first elasticity estimate;
receive, from the client computing device, an indication of a selection to access the client navigation system; and
generate, by the client navigation system, display instructions configured to display a user interface including a pre-service workflow prior to the client receiving the at least one upcoming service, wherein the user interface includes the first estimate and the first elasticity estimate

40. The computer-readable storage device of claim 39, wherein the computer readable instructions, when executed by the processing unit, are further configured to:
use the messaging processor to receive updated information corresponding to the at least one service or to the eligibility benefits data;
use the estimator to:
determine a second estimate based on the updated information; and
determine a second elasticity estimate associated with the second estimate; and
use the client navigation system to transmit a second notification to the client computing device, wherein the second notification includes a link to access the second estimate and the second change estimate.
